(19) United States
(12) Patent Application Publication
(10) Pub. No.: US 2003/0187325 A1
(43) Pub. Date: Oct. 2, 2003

(54) Medical laser treatment module

(76) Inventors: Jorg Meister, Korschenbroich (DE); Norbert Gutknecht, Roetgen (DE); Christian Apel, Aachen (DE)

Correspondence Address: CONNOLLY BOVE LODGE & HUTZ, LLP, 1220 N MARKET STREET, P O BOX 2207, WILMINGTON, DE, 19899

(21) Appl. No.: 10/362,134

(22) PCT Filed: Aug. 10, 2001
(86) PCT No.: PCT/DE01/02958

(30) Foreign Application Priority Data
Aug. 23, 2000 (DE) 10041421.4

(51) Int. Cl. A61B 1/00; A61B 1/04

(52) U.S. Cl. 600/108; 600/101

(57) ABSTRACT

(57) Abstract: The invention relates to a medical laser treatment module, which comprises a laser radiation source for generating a fundamental wavelength λ₁ and which is highly variable in use. According to the invention, the medical laser treatment module is characterized in that it comprises at least one means (2, 3) for generating laser radiation of another wavelength λ₁, λ₃ and at least one means for optionally injecting the laser radiation of the fundamental wavelength λ₁ into the means for generating the wavelength λ₂, λ₃. The laser module designed in such a manner can be used, in particular, in dentistry and both as an intergratable module and as a fixed component of a treatment unit.

The invention relates to a medical laser treatment module according to the generic part of claim 1.

Laser systems are indispensable tools in technology, in material processing as well as in medicine. They allow precise, point-accurate and contact-free work without mechanical wearing parts such as, for example, saw blades or drills.

Numerous laser systems exist for medical applications. An aspect of fundamental importance for each laser is its active medium since this is what determines the emission wavelengths and thus the area of application of the laser in medicine. This selection is made essentially on the basis of the wavelength-dependent absorption of laser radiation in the tissue.

Various laser systems are used in human medicine such as, for example, ophthalmology, dermatology, plastic surgery, gynecology, neurosurgery, urology and dentistry as well as in veterinary medicine. An example is the treatment of vision problems by means of an excimer laser whose emission spectrum lies in the ultraviolet range for correcting the cornea by removing minute quantities of tissue. Lasers are also used in the treatment of cataracts or glaucoma. In the treatment of glaucoma, the regulation of the intraocular pressure is restored. In dentistry, lasers are used, for example, to treat periodontitis and gum diseases as well as to replace drills.

The principle behind the generation of laser radiation is always stimulated emission, a process first described by Albert Einstein. Through the excitation of the atoms, or of the molecules, in the laser-active medium, higher energy levels are populated which are responsible for the laser transition. If the excitation is strong enough to generate (pump) an overpopulation of the upper laser level, this is referred to as a population inversion. Ultimately, due to a spontaneous emission transition to the stimulated emission, that is to say, to an artificially generated depopulation of the upper laser level, laser beams are radiated.

The process with which the laser medium is excited depends on the laser medium used. The three main types of excitation are:

i) gas discharge, that is to say, plasma formation in gas lasers;

ii) optical pumping in solid state laser systems;

iii) electric pumping in diode lasers.

Of central importance for the solid state laser is the laser-active medium that is contained therein and in which the laser radiation is generated. In case of solid state lasers, the laser-active medium is formed by a crystal that can be excited by means of various methods until the population inversion occurs.

Techniques known in the state of the art for the excitation of the laser crystal are, on the one hand, optical pumping with a flash lamp and, on the other hand, optical pumping with another laser system.

In the case of excitation by means of a flash lamp, part of the spectrum emitted by the flash lamp lies in the range of the absorption band of the laser crystal needed for the laser excitation. The crystal is excited by means of a transversal arrangement, i.e. the laser crystal and the flash lamp lie parallel to each other. The undesired heat output radiated by the flash lamp makes it indispensable to cool the laser crystal.

The excitation by means of another laser system can be carried out in various arrangement options:

1: The laser that is used to pump the crystal radiated in the longitudinal configuration, that is to say, along the lengthwise axis of the crystal.

2: An array of laser systems is arranged in the transversal configuration, that is to say, transversal to the crystal.

The advantage of the excitation of the laser medium by means of another laser is the narrow-band excitation of the laser transition by excited state absorption (ESC). In this process, as opposed to the broad-band excitation with a flash lamp, only a minimal amount of excitation energy is lost. However, one disadvantageous aspect is that the pumping energy, for example, in the case of the longitudinal excitation, is not uniformly distributed in the crystal.

Another process for generating the population inversion is so-called diffuse pumping. This process is disclosed in German patent application no. 100 13 371.1. The pump configuration used in this process cannot be described as being transversal or longitudinal. Rather, the pumping radiation for the crystal is coupled into the pumping chamber via special light transmission systems. Through multiple reflection on the inner wall surface of the pumping chamber, the laser crystal is homogeneously illuminated. The source of pumping light here can be made up of one or more lasers.

In the diode lasers, semiconductor crystals are used as the active media which, when excited, emit a coherent radiation in the visible and near-infrared spectral range. In semiconductors, the energy states of the electrons are not sharp as is the case with free atoms, but rather they are determined by broad bands. The valence band constitutes the ground (unexcited) state while the conduction band constitutes the excited state. The excitation normally takes place at the so-called p-n transition after an external voltage has been applied. The electrons are conveyed from the valence band into the conduction band, which leads to the population inversion. In a subsequent stimulated emission, they return to the valence band and emit light in the process. The emission wavelength depends on the energy gap between the valence band and the conduction band, whereby the band gap ensues from the selection of suitable semiconductor connections. As a rule, it is the elements from the second to fourth groups of the periodic table and/or mixed crystals from the third to fifth group that are of special importance.

It is the objective of the invention to create a laser device that can be used in medicine, that offers a wide array of application possibilities but that stands out for its compact design. The device should be easy to transport and to integrate as a modular building block into various devices.

Based on the generic part of claim 1, this objective is achieved by the features indicated in the characterizing part of claim 1.

With the medical treatment device, or rather the medical laser treatment device according to the invention, it is now possible to use just one device to carry out a large number of medical treatments that make different requirements of the wavelength of the laser radiation. Depending on the type of treatment, the desired wavelength can be generated with just one device.

Advantageous embodiments of the invention are presented in the subordinate claims.

The drawings show the mode of operation and the structure of the medical laser treatment device according to the invention in schematic form.

The following is shown in the drawings:

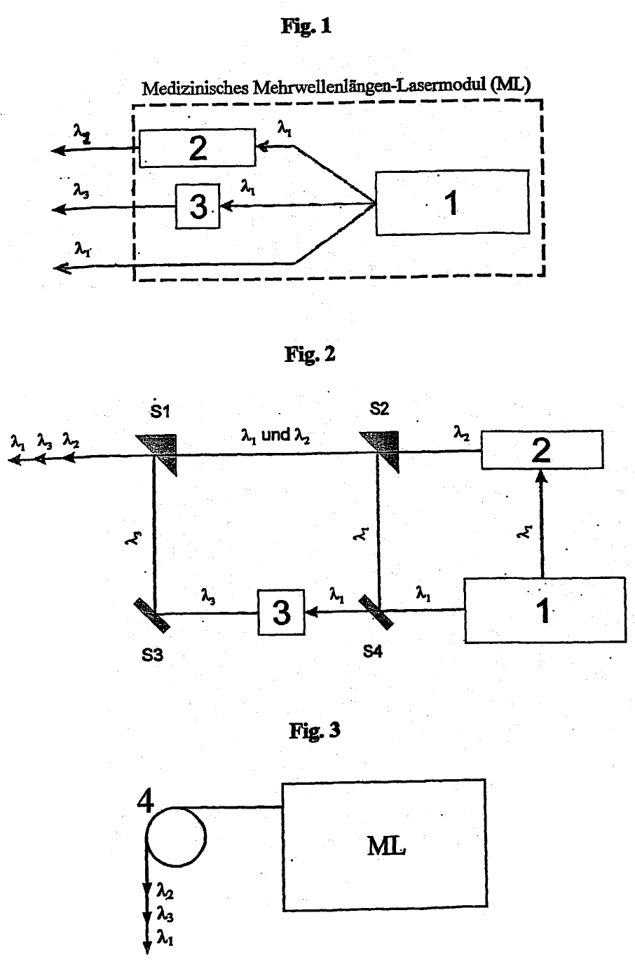

FIG. 1: a schematic representation of the mode of operation of the medical laser treatment device according to the invention;

FIG. 2: an exemplary embodiment of the invention;

FIG. 3: an overview of the medical laser treatment device.

In the block diagram shown in FIG. 1, the reference numeral 1 designates a diode laser, or a diode laser array. The embodiments relate to an individual diode laser as well as to a diode laser array. In the case of a diode laser array, the beams can be combined or conveyed individually, for example, by feeding them into optical fibers that are separate from each other. The solid state laser module is designated with the reference numeral 2 and a non-linear doubler unit with the reference numeral 3. The letter λ₂ designates the emission wavelength of the solid state laser 2, λ₃ is the emission wavelength of the non-linear doubler unit 3 and λ₁ is the emission wavelength of the diode laser 1.

It is particularly advantageous that the medical laser treatment module is equipped in such a way that it contains a light transmission system with a liquid light conductor. Preferably, the liquid light conductor serves to transmit various wavelengths over a very wide spectral range, as is the case in the embodiment of the multiple wavelength laser module.

The test arrangement shown in FIG. 2 shows an example of a set-up for beam superimposition. In this set-up, the diode laser 1 generates the emission wavelength λ₁, which is coupled into the solid state laser 2 which, in turn, emits the wavelength λ₂. The laser radiation having the wavelength λ₂ emitted by the solid state laser 2 traverses the semi-transparent mirrors S2 and S1 before leaving the laser module. Another partial beam having the wavelength λ₁ of the diode laser 1 strikes the beam divider S4, is reflected on the semi-transparent mirror S2 and likewise leaves the laser module, preferably so that the beam is superimposed with the beam having the wavelength λ₂ after the passage of the beam through the semi-transparent mirror S1. The other partial beam having the wavelength λ₁ generated at the beam divider S4 is coupled into the non-linear doubler unit where it is transformed into the wavelength λ₃. The beam having the wavelength λ₃ is reflected completely at the mirror 3 and leaves the laser module through reflection at the semi-transparent mirror S1, preferably so that the beam is superimposed with λ₁ and λ₂.

FIG. 3 shows how the superimposed beams having the wavelengths λ₁, λ₂ and λ₃ of a laser module ML are conducted to the destination site via a light transmission system 4 in a superimposed axis.

Below, the medical laser treatment module according to the invention will be described by means of several embodiments.

The medical laser treatment module is designed according to the invention in such a way that it has a laser radiation source 1 for generating a fundamental wavelength λ₁ and that it also has at least one means 2, 3 for generating laser radiation having an additional wavelength λ₂, λ₃, and at least one means for selectively coupling the laser radiation having the fundamental wavelength λ₁ into the means 2, 3 for generating the wavelengths λ₂, λ₃. Preferably, there are two means 2, 3 for generating the laser radiation having an additional wavelength λ₂, λ₃.

A plurality of means are suitable for coupling the fundamental wavelengths λ₁ into the means 2, 3. The version in which the means 2 for generating the laser radiation having the wavelength λ₂ is a solid state crystal is a preferred embodiment. However, the means for generating the laser radiation having the wavelength λ₂ can also be a different means. Preferably, this additional means for generating an additional wavelength is a doubler unit that generates laser radiation having the wavelength λ₃.

Suitable light transmission systems such as, for example, liquid light conductors or solid state fibers, especially glass fibers, serve to couple laser radiation into the means 2 and 3.

An alternative coupling in of the laser radiation is preferably done using suitable deflection systems that consist of suitable means such as mirror systems, beam dividers, dichroic mirrors or pivoting mirrors. The means for coupling in the laser radiation having the fundamental wavelength λ₁ can also comprise lens elements.

The coupling out of laser radiation from the means, 1, 2 and 3 for generating the laser radiation having the wavelengths λ₁, λ₂ and λ₃ preferably involves the means described above with respect to the coupling in of laser radiation.

Preferably, suitable light transmission systems, preferably using liquid light conductors or solid state fibers, are also employed for coupling out laser radiation.

Preferred deflection systems are suitable mirror systems, for example, beam dividers, dichroic mirrors or pivoting mirrors.

Preferably, prism or lens elements serve for purposes of coupling out.

In a preferred embodiment of the invention, the means 1 for generating the laser radiation having the wavelength λ₁ generates a shorter wavelength than the means 2 for generating the laser radiation having the wavelength λ₂ and a longer wavelength than the means 3 for generating the laser radiation having the wavelength λ₃.

Preferably, this is achieved in that a diode laser is used as the means 1 for generating the laser radiation having the wavelength λ₁, in that a solid state laser is used as the means 2 for generating the laser radiation having the wavelength λ₂ and a non-linear frequency doubler is used as the means 3 for generating the laser radiation having the wavelength λ₃.

The use of the diode laser as a means for generating the laser radiation having the wavelength λ₁, has, for one thing, the advantage that it is a very small component of the device according to the invention, which is highly advantageous for its use as a medical treatment device, especially for a portable treatment device. The wavelengths λ₁ generated by diode lasers can also be used directly for medical treatment. Thus, light of diode lasers in a wavelength range of 900 nm to 1000 nm can be used, preferably for treatment in the realm of periodontology, endodontics and surgery. This is of special significance in dentistry. Depending on the desired wavelengths, the diode laser 1 can have an active medium from the group consisting of gallium-arsenide (GaAs), indium-galliumarsenide (InGaAs), gallium-aluminum-arsenide (GaAlAs), indium-gallium-aluminumarsenide (InGaAlAs) or indium-gallium-arsenide-phosphite (InGaAsP). However, the selection is not limited to this group. Instead, any active medium can be used that is suitable for a medical treatment and/or that can serve to excite another laser which, in turn, is suitable for generating a medically usable wavelength λ₂, λ₃. A diode laser array can also be used instead of an individual diode laser 1.

In another preferred embodiment, the means 2 for generating the wavelength λ₂ can be a source of laser radiation that can generate laser radiation having the wavelength λ₂ in the range from 1.5 μm to 3 μm. Preferably, a solid state laser 2 can be used that has an active medium that is capable of generating the laser radiation having the wavelength λ₂ in a wavelength range from 1.5 μm to 3 μm. Examples of the active medium that can be used are crystals from the group consisting of Nd:YAG, Nd:YLF, Ho:YAG, Er:YAG, ErCr:YSGG, Er:GGG, Er:YSGG, Er:YLF, CrTmEr:YAG or crystals doped with other rare earths. However, the usable crystals are not limited to this group, but rather, any crystal can be used that is capable of generating laser radiation having a wavelength that is suitable for the medical treatment. The selection of the diode laser 1, or rather of the diode laser array for generating the fundamental wavelength λ₁ of the medical laser treatment device according to the invention depends on which solid state laser crystal is selected for generating the laser radiation having the wavelength λ₂. The crystals listed as examples for the solid state laser 2 are suitable for use with the diode laser crystals listed as examples for the diode laser 1. The laser radiation of the solid state laser 2 can be used, for example, for cavity preparation, periodontology, endodontics or for processing plastics.

When solid state lasers 2 are used as the means for generating the laser radiation having the wavelength λ₂, the solid state laser 2 is optically pumped by the means for generating the wavelength λ₁. This can be done by means of all kinds of pump mechanisms, but special preference is given to the process of diffuse pumping, especially the process described in the unpublished German patent application 100 13 371.1. In the case of diffuse pumping, the active medium of the solid state laser 2 is in a cavity that is mirrored at both of its ends, preferably over the broad sides and over the entire circumference. Preferably, the interior of the cavity is filled with a liquid that is likewise present in a line that couples the light having the wavelength λ₂ into the solid state laser 2. As a result, the interior of the cavity of the solid state laser 2 is homogeneously illuminated by light. Examples of possible liquids are aqueous solutions, silicone oils and/or other suitable liquids. In another preferred embodiment of the invention, the liquid used to couple the light having the wavelength λ₂ into the solid state laser 2 is conveyed in a circulation system that is equipped with a cooling aggregate. Thus, it is possible to cool the interior of the cavity.

The means 3 for generating the wavelength λ₃ is preferably a laser radiation source 3 that can generate laser radiation having the wavelength λ₃ in a wavelength range from 450 nm to 500 nm. Nonlinear-frequency doublers 3 are preferably used for this purpose into which optionally the light having the wavelength λ₁ is coupled in order to pump the frequency doubler crystal. Non-linear frequency doublers 3 with doubler crystals from the group consisting of KTP, KDP, LiNbO₃, KNbO₃, LiTaO₃ and LBO crystals have proven to be especially well-suited. With an additional periodical polarization, the performance spectrum of some crystals such as, for example, KTP or LiTaO₃, can be considerably enhanced. These crystals can generate laser radiation as a function of the pumping wavelength that can be used, for instance, in surgery, in endodontics and in the polymerization of plastics. However, the selection should not be limited to the examples, but rather, any crystal can be used that is capable of generating laser radiation having a wavelength that is suitable especially for medical applications. In a preferred embodiment, the non-linear frequency doubler is embedded in a resonator so that an additional amplification of its emission spectrum is possible.

According to the invention, the means for generating the wavelengths λ₁, λ₂, and λ₃ are incorporated in a module in such a way that at least one component from the group consisting of the wavelengths λ₁, λ₂ and λ₃ of the laser module can be conveyed through a light transmission system 4 at the treatment site. Examples of a possible light transmission system 4 are a fiberglass cable or any liquid-filled hollow structure (lumen) that conveys the light to a point of exit which then performs the medical treatment, controlled either by manual manipulation or purely mechanically. The wavelength necessary for the envisaged treatment is coupled into this light transmission system 4. The coupling in of the light having the various wavelengths can be carried out, for example, by arrangements of beam dividers S4, such as semi-transparent mirrors S1, S2 and mirrors S3.

An arrangement of module components depicted in FIG. 2 shows an example of an embodiment in which a diode laser 1 emits light having the wavelength λ₁, which is either conveyed directly via the beam divider S4 and the semi-transparent mirrors S2, S1 to a light transmission system 4, or else via the beam divider S4, to a non-linear frequency doubler 3, and then, after conversion into light having the wavelength λ₃ via the mirror S3 to the semi-transparent mirror S1, and finally to the light transmission system 4. Moreover, part of the light having the wavelength λ₁ is directly coupled out of the diode laser 1 and it then pumps a solid state laser 2 whose light having the wavelength λ₂ traverses the semi-transparent mirrors S2 and S1 and is fed to the light transmission system 4. Due to the fact that the wavelengths λ₂ and λ₃ are generated as a function of the wavelength λ₁, the output powers of λ₂ and λ₃ are directly coupled to the output line of λ₁. Therefore, the laser power at the wavelengths λ₁, λ₂ and λ₃ is set on the basis of the power of the fundamental wavelength λ₁, that is to say, in the present example, on the basis of the line adjustment of the diode laser 1 or of the diode laser array 1.

Application Examples:

Especially in dentistry, in the case of a preferred embodiment of the invention, the multiple wavelength laser module is used in the realms of cavity preparation, periodontology, surgery, endodontics and for the processing and polymerization of plastics. For this purpose, the following wavelengths can be used:

Cavity preparation: 2 μm to 3 μm, (λ₂)

Periodontology: 900 nm to 1000 nm as well as 2 μm to 3 μm, (λ₁ and λ₂).

Surgery: 450 nm to 500 nm, 900 nm to 1000 nm, (λ₁ and λ₃).

Endodontics: 450 nm to 500 nm, 900 nm to 1000 nm, 2 μm to 3 μm, (λ₁, λ₂ and λ₃).

Processing and polymerization of plastics: 2 μm to 3 μm, 450 nm to 500 nm, (λ₂ and λ₃).

Depending on the area of application in medicine, in a preferred embodiment of the invention to be used in dentistry, the provision is made for the emission spectrum to be selected as follows:

fundamental wavelength λ₁ of the diode laser, or of the diode laser array 1 in the range from 900 nm to 1000 nm,

wavelength λ₂ of the solid state laser 2 from 1.5 μm to 3 μm,

wavelength λ₃ of the doubler unit 3 from 450 nm to 500 nm.

Moreover, the medical laser treatment module according to the invention can be used in various areas in human medicine such as, for example, dermatology, ophthalmology or dentistry as well as in veterinary medicine.

The medical laser treatment module according to the invention makes it possible to generate several laser wavelengths inside a very small and compact structure that can be used as a stand-alone device, that is to say, as a completely independent unit, as a modular building block (selected wavelengths) or as an integratable module in the physician's practice. The selection of the laser wavelengths λ₁, λ₂ and λ₃ and thus the selection of the means for generating the laser radiation depend on the area of application in medicine. With a suitable beam arrangement, these can be conveyed out of the medical laser treatment module according to the invention either individually, in combination or in complete superimposition by means of a suitable light transmission system 4, as is shown in FIG. 3.

In a preferred embodiment of the medical laser treatment module according to the invention, it is provided to structure it as a stand-alone device. It contains all of the module components such as the diode laser or the diode laser array module 1 for generating the fundamental wavelength λ₁, the solid state laser module 2 for generating the low-frequency laser beams λ₂ and the doubler unit 3 for generating the laser wavelength λ₃. Furthermore, with a suitable beam arrangement, at least one component from the group consisting of wavelengths λ₁, λ₂ and λ₃ can be conveyed out of the medical laser treatment module according to the invention either individually or in complete superimposition. The beam is transported out of the device via one or more suitable light transmission systems 4.

In another preferred embodiment, it is provided to structure the device according to the invention as a modular building block. Here, a combination of individual module components is possible such as, for example, the diode laser or the diode laser array module 1 for generating the fundamental wavelength λ₁ and the solid state laser module 2 for generating the low-frequency laser beams having the wavelength λ₂, or the diode laser or the diode laser array module 1 for generating the fundamental wavelength λ₁ and the doubler unit 3 for generating the wavelength λ₃. Moreover, if suitably arranged, the laser wavelengths λ₃, λ₁ or λ₂, λ₁ can be conveyed out of a building block either individually or in complete superimposition, that is to say, λ₃ plus λ₁ or λ₂ plus λ₁, by means of a light transmission system 4.

In another preferred embodiment of the invention, the medical laser treatment module can be integrated as an integratable module, for example, into a dental treatment unit. This, in turn, can be done as a stand-alone system or as a modular building block. As far as the wavelength combination is concerned, all of the described variations of the stand-alone system or of the modular building block can be employed.

### LIST OF REFERENCE NUMERALS

1. Diode laser or diode laser array with λ₁: fundamental wavelength

2. Solid state laser module with λ₂: low-frequency emission spectrum

3. Doubler unit with λ₃: higher-frequency emission spectrum

4. Light transmission system

S1 Deflection prism, beam divider or mirror

S2 Deflection prism, beam divider or mirror

S3 Deflection prism, beam divider or mirror

S4 Deflection prism, beam divider or mirror

1. A medical laser treatment module comprising at least a first source of laser radiation (1) for generating a fundamental wavelength (λ₁) and at least one means (2, 3) for generating laser radiation having an additional wavelength (λ₂, λ₃) as well as a means for coupling the laser radiation having the fundamental wavelength (λ₁) into the means for generating the additional wavelengths (λ₂, λ₃), whereby in one operating state, the laser radiation having the fundamental wavelength (λ₁) as well as the laser radiation having at least one of the additional wavelengths (λ₂, λ₃) can be coupled out of the medical multiple wavelength laser module, whereby the wavelengths (λ₂ and λ₃) are generated as a function of the wavelength (λ₁), whereby the first source of laser radiation (1) is a diode laser, the means (2) for generating the wavelength λ₂ is a solid state laser and the means (3) for generating the wavelength λ₃ is a non-linear frequency doubler, and whereby, with a suitable beam arrangement, at least one component from the group consisting of wavelengths λ₁, λ₂ and λ₃ can be conveyed out of the medical laser treatment module either individually or in complete superimposition by means of a light transmission system.

2. The medical laser treatment module according to claim 1, characterized in that the solid state laser for generating the laser radiation having the wavelength (λ₂) generates a longer wavelength than (λ₁) and the non-linear frequency doubler for generating the laser radiation having the wavelength (λ₃) generates a shorter wavelength than (λ₁).

3. The medical laser treatment module according to one or both of claims 1 or 2, characterized in that the active medium of the diode laser is a component from the group consisting of gallium-arsenide (GaAs), indium-gallium-arsenide (InGaAs), gallium-aluminum-arsenide (GaAlAs), indium-gallium-aluminum-arsenide (InGaAlAs) or indium-gallium-arsenide-phosphite (InGaAsP).

4. The medical laser treatment module according to one or more of the preceding claims, characterized in that the diode laser is capable of generating light having a wavelength in the range λ₁ from 900 nm to 1000 nm.

5. The medical laser treatment module according to one or more of the preceding claims, characterized in that several diode lasers are arranged in a diode laser array.

6. The medical laser treatment module according to one or more of the preceding claims, characterized in that the solid state laser has a laser-active crystal from the group consisting of Nd:YAG, Nd:YLF, Ho:YAG, Er:YAG, ErCr:YSGG, Er:GGG, Er:YSGG, Er:YLF, CrTmEr:YAG or a crystal doped with other rare earths.

7. The medical laser treatment module according to one or more of the preceding claims, characterized in that the solid state laser is capable of generating a wavelength in the range from 1.5 μm to 3 μm.

8. The medical laser treatment module according to one or both of claims 6 or 7, characterized in that the crystal of the solid state laser is embedded in a cavity that allows diffuse pumping.

9. The medical laser treatment module according to claim 8, characterized in that the cavity of the solid state laser is connected to the source of laser radiation (1) via a liquid feed line in such a way that the light having the wavelength (λ₁) can be coupled into the cavity via the liquid in order to pump the crystal.

10. The medical laser treatment module according to claim 9, characterized in that the liquid feed line is configured as a circulation system that passes through a cooling aggregate.

11. The medical laser treatment module according to one or both of claims 9 or 10, characterized in that the liquid feed line and the cavity are filled with aqueous solutions, silicone oils and/or other suitable liquids.

12. The medical laser treatment module according to one or more of the preceding claims, characterized in that the non-linear frequency doubler has a doubler crystal from the group consisting of KTP, KDP, LiNbO₃, KNbO₃, LiTaO₃ and LBO.

13. The medical laser treatment module according to one or more of the preceding claims, characterized in that the non-linear frequency doubler generates wavelengths in the range of (λ₃) from 450 nm to 500 nm.

14. The medical laser treatment module according to one or more of the preceding claims, characterized in that the frequency doubler is equipped with a resonator in order to amplify its emission wavelength (λ₃).

15. The medical laser treatment module according to one or more of claims 1 to 14, characterized in that it contains a light transmission system with a liquid light conductor.

16. The medical laser treatment module according to one or more of claims 1 to 15, characterized in that it is a module that can be integrated into a medical treatment device.

17. A medical treatment instrument, characterized in that it has a medical laser treatment module according to one or more of claims 1 to 16.

18. The medical treatment instrument according to claim 17, characterized in that it is a dental treatment instrument.
